(19) United States
(12) Patent Application Publication
(10) Pub. No.: US 2009/0270669 A1
(43) Pub. Date: Oct. 29, 2009

(54) PROCESS FOR THE PREPARATION OF PROPYLENE FROM A HYDROCARBON FEED

(76) Inventors: Leslie Andrew Chewter, Amsterdam (NL); Michiel Johannes Franciscus Maria Verhaak, 1031 CM Amsterdam (NL); Jeroen Van Westrenen, Amsterdam (NL)

Correspondence Address: SHELL OIL COMPANY, P O BOX 2463, HOUSTON, TX, 772522463, US

(21) Appl. No.: 12/301,169

(22) PCT Filed: May 16, 2007
(86) PCT No.: PCT/EP07/54761

§ 371 Date: Dec. 9, 2008

(30) Foreign Application Priority Data
May 19, 2006 (EP) 06114281.6

(51) Int. Cl. C07C 4/02

(52) U.S. Cl. 585/653

(57) ABSTRACT

Process for the preparation of propylene from a hydrocarbon feed, wherein the hydrocarbon feed is an essentially olefinic hydrocarbon feed comprising C6 olefins and wherein the hydrocarbon feed is contacted with a one-dimensional zeolite having 10-membered ring channels and a silica to alumina ratio (SAR) in the range from 10 to 200.

### TECHNICAL FIELD OF THE INVENTION

This invention relates to a process for the preparation of propylene from a hydrocarbon feed.

### BACKGROUND OF THE INVENTION

Processes for the preparation of propylene from a hydrocarbon feed are well known in the art.

For example U.S. Pat. No. 6,307,117 describes a method for producing ethylene and propylene from a hydrocarbon feedstock by catalytic conversion over a zeolite containing catalyst. The hydrocarbon feed comprises 20% by weight or more, based on the total weight of the hydrocarbon feedstock of at least one C₄-C₁₂ olefin. The use of a group Ib metal promotor such as silver was found essential for a high yield conversion of the feed to ethylene and propylene. It was furthermore found necessary that the zeolites had a silica to alumina ratio in the range from 200 to 5000. Although, in passing zeolites such as ZSM-23 and ZSM-35 were mentioned, the use of such zeolites was not actually disclosed. The only zeolite used in the examples was ZSM-5.

In example 4 of U.S. Pat. No. 6,307,117 a 1-hexene feed was cracked into smaller components over a ZSM-5 catalyst having a silica to alumina ratio of 300. The product included propylene and butylene in a weight ratio of propylene to butylene of about 2.2 and propylene and ethylene in a weight ratio of propylene to ethylene of about 5.1.

WO-A-99/057226 describes a method for converting a hydrocarbon feedstock to propylene by contacting the hydrocarbon feedstock under cracking conditions with a catalyst selected from the group consisting of medium pore zeolites having a silica to alumina ratio in excess of 200. The olefinic hydrocarbon feedstock comprises from about 10% w/w to about 70% w/w olefins.

In example 1 of WO-A-99/057226, a 50/50 blend of n-hexane/n-hexene was contacted at 575° C. with a ZSM-48 catalyst and a ZSM-22 catalyst each having a silica to alumina ratio in excess of 1500. When using ZSM-22 according to the process of WO-A-99/057226, the product included propylene and butylene in a weight ratio of propylene to butylene of about 8.7 and propylene and ethylene in a weight ratio of propylene to ethylene of about 13.6. However, less than 50% of the feedstock was actually converted. In a comparative example in table 2 of WO-A-99/057226 a 50/50 blend of n-hexane/n-hexene was contacted at 575° C. with a ZSM-22 catalyst having a silica to alumina ratio of 120. This comparative example produced a product with a weight ratio of propylene to butylene of 2.0 and a weight ratio of propylene to ethylene of 3.8. at a conversion of 53% of the feedstock.

WO-A-01/81280 describes a process for cracking of C₄-C₉ feeds, preferably a feed consisting essentially of C₄ and/or C₅ olefins over a zeolite catalyst having one dimensional non-interconnecting channels, which may be selected from the group consisting of TON and MTT. The feed is cracked at a temperature in the range from 400 to 750° C. In the examples a butene/butane mixture is cracked over a MTT zeolite at a temperature of 526° C. These examples produce a product with a weight ratio of propylene to ethylene of at most 5.5 at a butene conversion of 68.9% w/w

WO-A-03/020667 describes a method of making olefins, comprising contacting an oxygenate feed with at least two different zeolite catalysts to form an olefin composition. In example 1 a hexene feed is contacted with ZSM-22 at a temperature of 650° C. The product produced contains propylene and ethylene in a ratio of 1.76.

It would be desirable to have a process that would be able to convert a hydrocarbon feedstock with a high conversion primarily into propylene.

### SUMMARY OF THE INVENTION

It has now been surprisingly found that an essentially olefinic hydrocarbon feed comprising C₆ olefins can be converted with high conversion primarily into propylene, even without the necessity for a Ib metal promoter, when a one-dimensional zeolite having 10-membered ring channels is used.

Accordingly, the present invention provides a process for the preparation of propylene from a hydrocarbon feed, wherein the hydrocarbon feed is an essentially olefinic hydrocarbon feed comprising C₆ olefins and wherein the hydrocarbon feed is contacted with a one-dimensional zeolite having 10-membered ring channels and a silica to alumina ratio (SAR) in the range from 10 to 200.

With the process according to the invention propylene can be prepared in a high selectivity with a high conversion.

### DETAILED DESCRIPTION OF THE INVENTION

By a hydrocarbon is understood a compound comprising both carbon atoms as well as hydrogen atoms. By a essentially olefinic hydrocarbon feed is understood a feed comprising hydrocarbons, which hydrocarbons consist essentially of olefins. By “consist essentially of” is understood that the hydrocarbon feed contains more than 80 wt %, more preferably more than 90 wt %, even more preferably more than 95 wt %, still more preferably more than 99 wt % and still even more preferably more than 99.9 wt % of a certain compound, in this case olefins, based on the total amount of hydrocarbons present. The remainder can be other hydrocarbons, for example saturated C₂-C₁₀ hydrocarbons and/or aromatic compounds. In one preferred embodiment the remainder consists essentially of saturated C₂-C₁₀ hydrocarbons, whilst aromatic compounds are absent. In another preferred embodiment the remainder consists essentially of other C₅-C₆ hydrocarbons, preferably saturated C₅-C₆ hydrocarbons.

Most preferably the hydrocarbon feed consists of only olefins.

The essentially olefinic hydrocarbon feed preferably comprises more than 20% w/w C₆ olefins, more preferably more than 30% w/w C₆ olefins and still more preferably more than 50% w/w C₆ olefins, based on the total amount of hydrocarbons present.

In a further preferred embodiment the hydrocarbon feed consists essentially of C₆ olefins or consists essentially of a mixture of C₆ and C₅ olefins. More preferably the hydrocarbon feed consists essentially of C₆ olefins. The wording “consist essentially of” is to be understood as defined above.

Preferably other hydrocarbons than C₆ and C₅ olefins are present in an amount of less than 20 wt %, more preferably less than 10 wt %, even more preferably less than 5 wt %, still more preferably less than 1 wt % and most preferably less than 0.1 wt %, based on the total amount of hydrocarbons present. In a preferred embodiment such other hydrocarbons comprise C₆ and C₅ saturated hydrocarbons. In a further preferred embodiment such other hydrocarbons are present in an amount from 0 to 500 ppmw. Most preferably the hydrocarbon feed consists 100% w/w of C₆ olefins or 100% w/w of C₅ and C₆ olefins, containing no detectable further compounds.

By a C₅ or a C₆ olefin is understood a hydrocarbon compound having 5 respectively 6 carbon atoms and having at least one double bond between two carbon atoms. Such an olefin can have one or two double bonds. Preferably the C₅ or C₆ olefin is a mono-olefin, having only one double bond.

Examples of suitable C₅ and C₆ olefins include n-pentene (e.g. 1-pentene or 2-pentene); cyclopentene; 2-methyl-butene (especially 2-methyl-2-butene); 3-methyl-1-butene; n-hexene (e.g. 1-hexene, 2-hexene or 3-hexene); cyclohexene; 2-methyl-pentene; 3-methyl-pentene; 2,3-dimethyl-1-butene; 2,3-dimethyl-2-butene. All possible cis and trans stereo-isomers of the various C₅ and C₆ olefin isomers can be used.

Preferably linear or branched, i.e. non-cyclic, C₅ and C₆ olefins are used.

In preferred embodiments the hydrocarbon feed can consist essentially of just C₆ olefins, or a mixture of C₆ and C₅ olefins. Most preferably the hydrocarbon feed consists only of C6 olefins.

Examples of suitable hydrocarbon feeds to the process include hydrocarbon streams derived from:

a C₆-hydrocarbon stream obtained after distillation from pyrolysis gasoline. Such a C₆-hydrocarbon stream (i.e. a stream containing hydrocarbons having 6 carbon atoms) can be partly hydrogenated and benzene is extracted therefrom, before use in the process of the invention;

a (part of a) C₆-hydrocarbon stream obtained from a reformer;

mixtures of any of the above or a mixture of any of the above with a C₅ and/or C₆ hydrocarbon stream obtained from another source.

The hydrocarbon feed is contacted with a one-dimensional zeolite having 10-membered ring channels and a silica to alumina ratio in the range from 1 to 200.

The zeolite is a one-dimensional zeolite having 10-membered ring channels. These are understood to be zeolites having only 10-membered ring channels in one direction which are not intersected by other 8, 10 or 12-membered ring channels.

Preferably the zeolite is selected from the group of TON-type (for example ZSM-22), MTT-type (for example ZSM-23) and EU-2/ZSM-48 zeolites.

The zeolites used in the present invention are distinct from zeolites having small pore 8-ring channels or zeolites having large pore 12-ring channels.

MTT-type catalysts are more particularly described in e.g. U.S. Pat. No. 4,076,842. For purposes of the present invention, MTT is considered to include its isotypes, e.g., ZSM-23, EU-13, ISI-4 and KZ-1.

TON-type zeolites are more particularly described in e.g. U.S. Pat. No. 4,556,477. For purposes of the present invention, TON is considered to include its isotypes, e.g., ZSM-22, Theta-1, ISI-1, KZ-2 and NU-10.

EU-2-type zeolites are more particularly described in e.g. U.S. Pat. No. 4,397,827. For purposes of the present invention, EU-2 is considered to include its isotypes, e.g., ZSM-48.

In a further preferred embodiment a zeolite of the MTT-type, such as ZSM-23, is used.

Preferably a zeolite in the hydrogen form is used, e.g., HZSM-22, HZSM-23, HZSM-48. Preferably at least 50% w/w, more preferably at least 90% w/w, still more preferably at least 95% w/w and most preferably 100% of the total amount of zeolite used is zeolite in the hydrogen form. When the zeolites are prepared in the presence of organic cations the zeolite may be activated by heating in an inert or oxidative atmosphere to remove the organic cations, for example, by heating at a temperature over 500° C. for 1 hour or more. The hydrogen form can then be obtained by an ion exchange procedure with ammonium salts followed by another heat treatment, for example in an inert or oxidative atmosphere at a temperature over 500° C. for 1 hour or more. The zeolites obtained after ion exchange with ammonium salts are also referred to as being in the ammonium form.

Preferably the zeolite has a silica to alumina ratio (SAR) in the range from 10 to 200. More preferably a zeolite having a SAR in the range from 10 to 100 is used.

The zeolite can be used as such or in combination with a so-called binder material. The zeolite as such or the zeolite in combination with a binder material, are hereafter both also referred to as zeolite catalyst or catalyst.

It is desirable to provide a zeolite catalyst having good mechanical strength, because in an industrial environment the catalyst is often subjected to rough handling which tends to break down the catalyst into powder-like material. The later causes problems in the processing. Preferably the zeolite is therefore incorporated in a binder material. Examples of suitable binder materials include active and inactive materials and synthetic or naturally occurring zeolites as well as inorganic materials such as clays, silica, alumina, aluminosilicate. For present purposes, inactive materials of a low acidity, such as silica, are preferred because they may prevent unwanted side reactions which may take place in case a more acidic material, such as alumina is used. Preferably the catalyst used in the process of the present invention comprises, in addition to the zeolite, 2 to 90 wt %, preferably 10 to 85 wt % of a binder material.

The process of the present invention can be carried out in a batch, continuous, semi-batch or semi-continuous manner using conventional reactor systems such as fixed bed, moving bed, fluidized bed and the like. As a reactor any reactor known to the skilled person to be suitable for catalytic cracking can be used.

Conventional catalyst regeneration techniques can be employed. The catalyst used in the process of the present invention can have any shape known to the skilled person to be suitable for this purpose, for example the catalyst can be present in the form of catalyst tablets, rings, extrudates, etc. extruded catalysts can be applied in various shapes, such as, cylinders and trilobes. If desired, spent catalyst can be regenerated and recycled to the process of the invention.

Preferably the hydrocarbon feed is contacted with the zeolite at a temperature in the range from 300 to 550° C. to effect cracking of the hydrocarbon feed. By cracking of the hydrocarbon feed is understood the effective cracking hydrocarbons into smaller hydrocarbons. More preferably the hydrocarbon feed is contacted with the zeolite catalyst at a temperature in the range from 400° C. to 550° C., and still more preferably in the range from 450° C. to 550° C.

The pressure can vary widely, preferably a pressure in the range from 1 to 5 bar is applied.

The hydrocarbon feed may be diluted with a diluent gas. Any diluent gas known by the skilled person to be suitable for such purpose can be used. Examples of a diluent gas include argon, nitrogen and steam. For example, the hydrocarbon feed can be diluted with steam, for example in the range from 0.01 to 10 kg steam per kg hydrocarbon feed.

The process according to the invention can advantageously be carried out in the absence of any metals belonging to Group Ib of the periodic table. By the absence of Group Ib metals is understood that, if present, the weight percentage of Group Ib metals on total amount of the zeolite is less than 0.1% w/w, more preferably less than 0.01% w/w, even more preferably less than 50 ppmw still more preferably less than 10 ppmw and most preferably non-existent.

Preferably the process is carried out in the absence of oxygenates. By the absence of oxygenates is understood that, if present, the weight percentage of oxygenates on total amount of the hydrocarbon feed is less than 5% w/w, more preferably less than 1% w/w, even more preferably less than 0.1% w/w, still more preferably less than 0.01% w/w and most preferably non-existent.

With the process according to the invention primarily propylene can be prepared with a high conversion.

A product stream of propylene can be separated from the reaction product by any method known to the person skilled in the art. Preferably such a separation is carried out in one or more distillation columns.

Depending on the hydrocarbon feed used, the reaction product can further contain unreacted C₅ and/or C₆ olefins. Such unreacted olefins are preferably recycled.

The process of the invention will herein below be illustrated by a number of non-limiting examples.

### Example 1 and Comparative Example A

In this example 1-hexene was reacted respectively over a MTT-type (according to the invention) and a MFI-type zeolite (comparative). The silica-to-alumina ratio were 48 and 280 for the MTT-type zeolite and the MFI-type zeolite, respectively. A sample of zeolite powder was pressed into tablets and the tablets were broken into pieces and sieved. For catalytic testing, the sieve fraction of 30-80 mesh has been used. A quartz reactor tube of 3 mm internal diameter was loaded with 200 mg of sieve fraction. Prior to reaction, the fresh catalyst in its ammonium-form was treated with flowing argon at 550° C. for 1 hour. Next, the catalyst was cooled in argon to the reaction temperature and a mixture consisting of 2.0 vol. % 1-hexene and 1 vol. % of water (in Argon) was passed over the catalyst at atmospheric pressure (1 bar) at a flow rate of 50 ml/minute. Periodically, the effluent from the reactor was analyzed by gas chromatography (GC) to determine the hydrocarbon product composition, based on the total hydrocarbon effluent of the reactor. The hydrocarbon product composition has been calculated on a weight basis. The following table (Table 1) lists the reaction parameters together with the product composition, as determined by Gas Chromatography:

TABLE 1

| | | MFI-type |
|---|---|---|
| Zeolite | MTT-type | (comparative) |
| Time on stream, hours | ~20 | ~20 |
| Temperature ° C. | 525° C. | 525° C. |
| 1-hexene conversion, wt % | ~100 | ~100 |
| Ethylene, wt % | 3 | 8 |
| Propylene, wt % | 88 | 74 |
| Butene isomers, wt % | 5 | 12 |
| Pentene isomers, wt % | <1 | 2 |
| propylene/ethylene ratio | 29 | 9 |
| propylene/butylene ratio | 18 | 6 |

As conversion is complete, the mass-based selectivity has the same value as the wt %.

### Comparative Example B

In this example 2-methyl-2-butene was reacted over an MTT-type zeolite. The silica-to-alumina ratio of the MTT-type zeolite was 48. A sample of zeolite powder was pressed into tablets and the tablets were broken into pieces and sieved. For catalytic testing, the sieve fraction of 30-80 mesh has been used. A quartz reactor tube of 3 mm internal diameter was loaded with 200 mg of sieve fraction. Prior to reaction, the fresh catalyst in its ammonium-form was treated with flowing argon at 550° C. for 1 hour. Next, the catalyst was cooled in argon to the reaction temperature and a mixture consisting of 1.5 vol. % 2-methyl-2-butene (2M2B) and 1 vol. % of water in Argon was passed over the catalyst at atmospheric pressure (1 bar) at a flow rate of 50 ml/minute. Periodically, the effluent from the reactor was analyzed by gas chromatography (GC) to determine the product composition. The composition has been calculated on a weight basis. The selectivity has been defined by the division of the mass of product i by the sum of the masses of all products. The following table (Table 2) lists reaction parameters together with the compositional data, as determined by GC:

TABLE 2

| Zeolite | MTT |
|---|---|
| Time on stream, hours | ~24 |
| Temperature ° C. | 525° C. |
| 2M2B conversion, wt % | 92 |
| Ethylene, wt. %/selectivity, % | 33/36 |
| Propylene, wt. %/selectivity, % | 49/54 |
| Butene isomers, wt. %/selectivity, % | 4/4 |
| propylene/ethylene ratio | 1.5 |
| propylene/butylene ratio | 13.5 |

(in this example the use of selectivity instead of wt. % product composition gives similar numbers because the conversion of C5 is close to 100%).

### Example 2

In this example 1-hexene was reacted over TON and MTT type zeolites at two space velocities. The silica-to-alumina ratio were 102 and 48 for TON and MTT, respectively. A sample of zeolite powder was pressed into tablets and the tablets were broken into pieces and sieved. For catalytic testing, the sieve fraction of 40-60 mesh has been used. A quartz reactor tube of 3 mm internal diameter was loaded with either 50 or 200 mg of this sieve fraction. Prior to reaction, the fresh catalyst in its ammonium-form was treated with flowing argon at 550° C. for 2 hours. Next, the catalyst was cooled in argon to the reaction temperature and a mixture consisting of 2.6 vol. % 1-hexene and 2 vol. % of water in Argon, was passed over the catalyst at atmospheric pressure (1 bar) at a flow rates of 50 ml/min (200 mg catalyst) and 100 ml/min (50 mg catalyst). Gas hourly space velocities (GHSV) are 15,000 and 120,000 ml/gram/hr, respectively, based on total gas flow. All Gas Hourly Space Velocities are measured at standard temperature and pressure (STP), i.e. at 23° C. and 1 bar. Weight hourly space velocities (WHSV) are 1.5 and 11.7 gram hexene/gram catalyst/hr, based on hexene mass flow. Periodically, the effluent from the reactor was analyzed by gas chromatography (GC) to determine the product composition. The composition has been calculated on a weight basis. The following table (Table 3) lists reaction parameters together with the compositional data, as determined by GC:

TABLE 3

| | MTT | TON | MTT | TON |
|---|---|---|---|---|
| GHSV STP | 120,000 | 120,000 | 15,000 | 15,000 |
| (ml/gram/hr-1) | | | | |
| WHSV (gram/gram/hr-1) | 11.7 | 11.7 | 1.5 | 1.5 |
| Temperature ° C. | 500° C. | 500° C. | 500° C. | 500° C. |
| 1-hexene conversion, | ~100 | ~100 | ~100 | ~100 |
| wt % | | | | |
| Ethylene, wt. % | 2.1 | 2.3 | 5.1 | 5.3 |
| Propylene, wt. % | 93.3 | 92.5 | 84.2 | 83.3 |
| Butene isomers, wt. % | 4.1 | 4.5 | 8.8 | 9.2 |
| Pentene isomers, wt. % | 0.4 | 0.5 | 1.1 | 1.1 |
| Propylene to Ethylene | 44.4 | 40.2 | 16.5 | 15.7 |
| weight ratio | | | | |
| Propylene to Butene | 22.8 | 20.6 | 9.6 | 9.1 |
| weight ratio | | | | |

Selectivity (based on weight) is the same as feed composition, in wt. %, since conversion levels are ˜100%.

### Example 3 and Comparative Example C

In this example a mixture of 1-hexene and n-hexane was reacted over a MTT zeolite and compared to that with a feed of pure 1-hexene. The silica-to-alumina ratio of MTT was 48. A sample of zeolite powder was pressed into tablets and the tablets were broken into pieces and sieved. For catalytic testing, the sieve fraction of 40-60 mesh has been used. The fresh catalyst in its ammonium-form was first treated in air at 600° C. for 4 hours. A quartz reactor tube of 3 mm internal diameter was loaded with 50 mg of catalyst. The reactor was heated in argon to the reaction temperature and either a mixture consisting of 2.2 vol. % 1-hexene, 1.8 vol % n-hexane 2 vol. % of water or consisting of 4.5% 1-hexene and 2 vol. % of water was passed over the catalyst at atmospheric pressure at a flow rates of 100 ml/min. Gas hourly space velocity (GHSV) is 120,000, based on total gas flow. Weight hourly space velocities (WHSV) is 18 gram(hexene+hexane)/gram catalyst/hr, based on combined (hexene+hexane) mass flow. Periodically, the effluent from the reactor was analyzed by gas chromatography to determine the product composition. The selectivity has been defined by the division of the mass of product i by the sum of the masses of all products. The following table (table 4) lists some of most important reaction parameters together with the compositional data, as determined by GC:

TABLE 4

| | MTT | MTT * |
|---|---|---|
| Hexane:Hexene | 0 | 1 |
| GHSV, ml/gram/hr | 120,000 | 120,000 |
| Temperature ° C. | 525° C. | 525° C. |
| 1-hexene conversion, wt % | ~100 | ~100 |
| n-hexane conversion, wt % | — | 14 |
| Ethylene, wt. % | 2.6 | 3.4 |
| Propylene, wt. % | 91 | 90 |
| Butene isomers, wt. % | 5.1 | 6.1 |
| Pentene isomers, wt. % | 0.9 | 0.8 |
| Propylene to Ethylene weight ratio | 35 | 26.5 |
| Propylene to Butene weight ratio | 17.8 | 14.8 |

* = Comparative

### Example 4 and Comparative Example D

In this example 1-hexene was reacted respectively over a TON-type zeolite having a silica-to-alumina ratio (SAR) of 130 (according to the invention) a TON-type zeolite having a silica-to-alumina ratio (SAR) of 250 (comparative). A mixture consisting of 2 vol. % 1-hexene and 1 vol. % of water (in Argon) was passed over 50 g catalyst at atmospheric pressure (1 bar) at a flow rate of 100 ml/minute. The effluent from the reactor was analyzed by gas chromatography (GC) to determine the product composition. The selectivity has been defined by the division of the mass of product i by the sum of the masses of all products. The following table (Table 5) lists the reaction parameters together with the product composition, as determined by Gas Chromatography:

TABLE 5

| SAR of TON-type zeolite | 130 | 250 (comparative) |
|---|---|---|
| Temperature ° C. | 500° C. | 500° C. |
| 1-hexene conversion, wt % | 97 | 66 |
| Ethylene, selectivity | 2 | 2 |
| Propylene, selectivity | 93 | 91 |
| Butene isomers, selectivity | 4 | 4 |
| Pentene isomers, selectivity | 1 | 3 |
| propylene/ethylene ratio | 47 | 46 |
| propylene/butylene ratio | 23 | 23 |

As can be seen from the above, the use of a TON-type zeolite with a SAR in the range from 10 to 200 results in a high conversion primarily into propylene.

1. A process for the preparation of propylene from a hydrocarbon feed, wherein the hydrocarbon feed is a hydrocarbon feed containing more than 80 wt % olefins, based on the total amount of hydrocarbons present, and comprising C₆ olefins and wherein the hydrocarbon feed is contacted with a one-dimensional zeolite having 10-membered ring channels and a silica to alumina ratio (SAR) in the range from 10 to 200, wherein at least 50% w/w of the total amount of zeolite used is zeolite in the hydrogen form.

2. The process according to claim 1, wherein the zeolite has a silica to alumina ratio in the range from 10 to 100.

3. The process according to claim 1, wherein the process is carried out at a temperature in the range from 300 to 550° C.

4. The process according to claim 1, wherein the hydrocarbon feed contains more than 80 wt % of C₆ olefins, based on the total amount of hydrocarbons present.

5. The process according to claim 1, wherein the hydrocarbon feed contains more than 80 wt % of a mixture of C₆ and C₅ olefins, based on the total amount of hydrocarbons present.

6. The process according to claim 1, wherein the zeolite is chosen from TON-type, MTT-type and EU-2/ZSM-48 zeolites.

7. The process according to claim 1, wherein the zeolite is a MTT-type zeolite.

8. The process according to claim 1, wherein the zeolite is a TON-type zeolite.

9. The process according to claim 1, wherein at least part of any unconverted feed is recycled.
